(19) United States
(12) Patent Application Publication
(10) Pub. No.: US 2026/0240482 A1
(43) Pub. Date: Aug. 20, 2026

(54) Implantable Medical Device Configured to Store Snapshots of Ectopic Events

(71) Applicant: BIOTRONIK SE & Co. KG, Berlin (DE)

(72) Inventors: R. Hollis WHITTINGTON, Portland, OR (US); Ravi Kiran Kondama REDDY, Portland, OR (US); Joseph Theodore MARMERSTEIN, Hudson, OH (US); Patrick L. PARISH, Portland, OR (US); Dirk MUESSIG, West Linn, OR (US); Hannes KRAETSCHMER, West Linn, OR (US)

(73) Assignee: BIOTRONIK SE & Co. KG, Berlin (DE)

(21) Appl. No.: 19/164,986

(22) PCT Filed: Mar. 6, 2024
(86) PCT No.: PCT/EP2024/055781

§ 371 Date: Sep. 12, 2025

Related U.S. Application Data
(60) Provisional application No. 63/454,192, filed on Mar. 23, 2023.

(30) Foreign Application Priority Data
Apr. 19, 2023 (EP) 23168651.0

(51) Int. Cl. A61B 5/364; A61B 5/00; A61B 5/02; A61B 5/287; A61B 5/333

(52) U.S. Cl. CPC A61B 5/364; A61B 5/0006; A61B 5/0013; A61B 5/02028; A61B 5/287; A61B 5/333

(57) ABSTRACT

An implantable medical device for sensing electrocardiogram signals includes an arrangement of electrode poles configured to sense electrocardiogram signals, and a processing module for processing electrocardiogram signals obtained by the arrangement of electrode poles. The processing module is configured to identify a contraction event based on said electrocardiogram signals, classify said contraction event as an ectopic event based on a relation to other contraction events prior and/or subsequent to said contraction event, and store a snapshot representing data of said electrocardiogram signals in a time period encompassing said contraction event classified as an ectopic event.

### CROSS-REFERENCE TO RELATED APPLICATIONS

This application is the United States National Phase under 35 U.S.C. § 371 of PCT International Patent Application No. PCT/EP 2024/055781, filed on Mar. 6, 2024, which claims the benefit of European Patent Application No. 23168651.0, filed on Apr. 19, 2023, and U.S. Provisional Patent Application No. 63/454,192, filed on Mar. 23, 2023, the disclosures of which are hereby incorporated by reference herein in their entireties.

### TECHNICAL FIELD

The present invention generally relates an implantable medical device for sensing electrocardiogram signals and to a method for operating such an implantable medical device.

### BACKGROUND

An implantable medical device of the type concerned herein comprises an arrangement of electrode poles configured to sense electrocardiogram signals and a processing module configured for processing electrocardiogram signals obtained by the arrangement of electrode poles.

An implantable medical device of this kind may for example be a pacemaker, an implantable pulse generator (IPG), an implantable cardioverter defibrillator (ICD), a sensor device such as a bio-sensor, or a monitoring device such as an implantable cardiac monitor (ICM). The implantable medical device herein is configured to sense electrocardiogram signals.

For example, the implantable medical device may be a monitoring device which is configured to record electrocardiogram signals and to communicate recorded electrocardiogram signals or information derived from recorded electrocardiogram signals to an external device in the context of a home monitoring system.

An implantable medical device as for example described in EP 3 278 836 B1 may for example comprise a housing and an arrangement of electrode poles arranged on the housing. The electrode poles herein are arranged on the housing of the implantable medical device such that the electrode poles are aligned along a longitudinal axis along which the implantable medical device extends. The electrode poles may for example be formed by housing segments which are made from an electrically conductive material such as a metal material and are exposed to the outside such that they may be brought into electrical contact with surrounding tissue in order to establish an electrical coupling to the tissue in an implanted state of the implantable medical device.

An implantable medical device as e.g. used in a home monitoring system shall allow for a reliable monitoring of a physiological state of a patient. In particular, using the implantable medical device it shall be possible to reliably detect an abnormal cardiac state based on recorded electrocardiogram signals. If an abnormality is detected in electrocardiogram signals, the implantable medical device shall be enabled to communicate with for example an external device of a home monitoring system, e.g. in order to trigger a message to a service center to alert medical personnel of a potential need for attention.

An abnormal cardiac state may in particular relate to the occurrence of ectopic events. Ectopic events may occur as so-called premature ventricular contraction events, premature atrial contraction events, or so called interpolated premature ventricular contraction events. Whereas premature ventricular contraction events can generally be assumed to be due to irregular contractions stemming from the ventricles, premature atrial contraction events have their origin in the atria of the patient's heart. Ectopic events originating from other foci within the heart are also possible and may be classified as junctional beats or other names. The common property of ectopic events is that they originate from an abnormal location.

In particular premature ventricular contractions (in short PVCs; also referred to as premature ventricular complexes, premature ventricular beats, premature ventricular depolarizations, or ventricular extrasystoles) may pose a risk for a patient, especially if premature ventricular contractions occur frequently, potentially indicating a risk for developing irregular heart rhythms (arrhythmias) or a beginning of disease of the heart muscle (cardiomyopathy). Premature ventricular contraction events are generally triggered from the ventricular myocardium and can be associated with structural heart disease and with many forms of cardiac disease, independent of severity. Premature ventricular contractions may be a precursor of cardiac pathology, but are also known to accompany extra-cardiac pathologies such as uncontrolled hypertension, thyroid dysfunction, pulmonary disease and sleep apneas.

There is a general desire for diagnostic tools allowing for the assessment of the occurrence of ectopic events in a patient, in order to help to provide a proper treatment at the right time to a patient for reducing a risk associated with a high burden of ectopy in a patient.

The present disclosure is directed toward overcoming one or more of the above-mentioned problems, though not necessarily limited to embodiments that do.

### SUMMARY

It is an object to provide an implantable medical device and a method for operating such an implantable medical device which in a reliable manner allow for a monitoring of a patient's cardiac condition over a prolonged period of time.

In one aspect, an implantable medical device for sensing electrocardiogram signals comprises an arrangement of electrode poles configured to sense electrocardiogram signals and a processing module for processing electrocardiogram signals obtained by the arrangement of electrode poles. The processing module is configured to identify a contraction event based on said electrocardiogram signals, classify said contraction event as an ectopic event based on a relation to other contraction events prior and/or subsequent to said contraction event, and store a snapshot representing data (e.g. a signal portion) of said electrocardiogram signals in a time period encompassing a contraction event classified as an ectopic event. The data represented by the snapshot may be at least one signal portion, sample time series data, and/or trends of intervals or amplitudes.

Ectopic events generally may occur as premature ventricular contraction events (PVCs), premature atrial contraction events (PACs), our so-called interpolated premature ventricular contraction events.

Premature ventricular contraction properties can be highly variable, depending on the origin, location and timing of the ventricular depolarization, but generally share two characteristics. A first characteristic is abnormal timing: Premature ventricular contractions occur prematurely, faster than the predominant sinus rhythm, and are typically followed by a compensatory pause due to retrograde block in the AV node preventing conduction of the next P-wave. A second characteristic relates to ECG morphology: The shape of a waveform relating to a premature ventricular contraction event is dependent on the origin location of the premature ventricular contraction, but the morphology is generally abnormal, and can be recognized when compared to a normally conducted beat.

Premature atrial contractions in turn generally occur at an abnormal, premature timing, but are similar in their morphological shape to a regular heartbeat in an electrocardiogram signal. For premature atrial contractions, hence, an ECG signal morphology will not differ significantly from an ECG signal morphology of a regular heartbeat, but will come at an abnormal timing in relation to a regular heartbeat.

Interpolated premature ventricular contractions typically occur as extra beats in between two regular heartbeats, without a change in the sinus rhythm, such interpolated premature ventricular contractions however exhibiting a significantly different morphology in comparison to a regular heartbeat.

The implantable medical device comprises a processing module which is configured to process sensed electrocardiogram signals in order to identify contraction events within the electrocardiogram signals and classify contraction events as ectopic events by processing signal portions of the electrocardiogram signals indicative of a particular contraction event in relation to other contraction events. The contraction events identified in the electrocardiogram signals generally are due to ventricular activity, which is typically visible as so-called QRS complexes in the electrocardiogram signals.

Based on the occurrence of one or multiple ectopic events, herein, the processing module of the implantable medical device is configured to store a snapshot representing data (e.g. a signal portion) of electrocardiogram signals in a time period encompassing one or multiple contraction events classified as ectopic events. Hence, based on the identification of ectopic events within electrocardiogram signals, a snapshot representation of the electrocardiogram signals, in particular of a signal portion indicative of ectopic events, is stored within the implantable medical device for future use.

The snapshot in particular may be saved in a permanent memory of the implantable medical device. The snapshot consists of a time series of electrocardiogram signal data from one or more electrocardiogram channels as recorded by the implantable medical device, for example stemming from one or multiple different pairs of electrode poles on one or multiple electrode leads or on a device body. The snapshot may be communicated, as a data representation, to an external device, wherein the data transfer to the external device may take place immediately upon storing the snapshot or may take place within a regular, periodic reporting at a fixed time or time interval, or the snapshot may be stored within the implantable medical device for a later download or review by an external device.

For example, over the lifetime of the implantable medical device, which may range from a multiplicity of months to a multiplicity of years and during which the implantable medical device rests in an implanted state within the patient, the implantable medical device senses electrocardiogram signals and, based on the sensed electrocardiogram signals, identifies ectopic events in the electrocardiogram signals. Based on the detection of ectopic events, continuously over the lifetime of the implantable medical device snapshots may be stored and may be communicated to an external device, such that based on the occurrence of ectopic events snapshot representations of signal portions of electrocardiogram signals encompassing these detected ectopic events are obtained.

Based on such snapshots, electrocardiogram signals relating to ectopic events may be reviewed and diagnosed by clinical personnel to initiate proper treatment to a patient.

The processing module of the implantable medical device is configured to store a snapshot representing data (e.g. a signal portion) of electrocardiogram signals in a time period encompassing one or multiple contraction events classified as one or multiple ectopic events and further including e.g. regular heart beats. The time period may have a length between 10 seconds to 24 hours, in particular between 30 seconds to 10 minutes, for example 1 minute. If the time period has a length of 1 minute, the snapshot encompasses a time series of electrocardiogram signal data within the time period of 1 minute, such that a signal portion of the electrocardiogram signals of a length of 1 minute is stored for future processing, in particular for communication to an external device.

The length of the time period for which the snapshot is stored may for example be programmable by a user and hence may be fixed within the system. Alternatively, the snapshot length may be dynamically adapted for example in order to encompass a particular series of ectopic events. The snapshot event may also contain processed or summarized data such as intervals of heart beats only and/or amplitudes and not the full ECG signal. The summarized data may reduce the storage requirement and provide the necessary information for diagnosis and decision on clinical course of action.

In one embodiment, the processing module is configured to derive at least one occurrence measure indicative of an occurrence of at least one ectopic event and to store the snapshot based on the at least one occurrence measure. The storing of the snapshot generally may be triggered based on certain criteria. For evaluating such criteria, an occurrence measure may be determined, the occurrence measure for example being indicative of the number of ectopic events in a certain time period or of the type of ectopic events that have occurred. Based on the occurrence measure it is determined whether a snapshot shall be stored, such that the occurrence measure serves to evaluate a trigger criterion for storing the snapshot.

Based on a trigger condition determined according to the occurrence measure, herein, a future storing of a snapshot may be triggered, the snapshot hence encompassing a data series of electrocardiogram signal data of a future time period after a trigger condition has occurred. Additionally or alternatively, the storing of a snapshot encompassing a time series of electrocardiogram signal data of a past time period encompassing the occurrence of past ectopic events may be triggered.

In one embodiment, the processing module is configured to derive the at least one occurrence measure to be indicative of the occurrence of at least one ectopic event in a predefined time interval, the occurrence of at least one further ectopic event after a prior ectopic event in a predefined time interval, the occurrence of a number of ectopic events in a predefined time interval equal to or larger than a predefined threshold, and/or the occurrence of an ectopic event of a predefined morphological class or of a shape different than a predefined morphological class.

In particular, it may be determined whether within a certain time interval one or multiple ectopic events occur. For this, the occurrence measure may be determined e.g. as a Boolean parameter indicating the presence of one or multiple ectopic events in a certain time interval, or may be determined to indicate an ectopic burden indicative of the number of ectopic events within the time interval. The time interval may be equal in temporal length to the time period of the snapshot or may be longer than the time period of the snapshot.

For example, for determining the occurrence measure it may be determined whether within a predefined time interval one or multiple further ectopic events may occur after a first ectopic event. If a single ectopic event in a time interval is detected, this may by itself not yet trigger the storing of a snapshot. However, if one or multiple further ectopic events, for example a sequence of ectopic events indicative of a run of ectopic events, are detected within the time interval, this may trigger the storing of the snapshot. The time interval may be equal in temporal length to the time period of the snapshot or may be longer than the time period of the snapshot.

In another example, the storing of the snapshot may be triggered if the number of ectopic events in a predefined time interval is larger than a predefined threshold. The time interval may be equal in temporal length to the time period of the snapshot or may be longer than the time period of the snapshot. For example, as an occurrence measure, the number of ectopic events per minute, per hour or per day may be determined in order to derive an activity burden per minute, per hour or per day, and based on the occurrence measure the storing of one or multiple snapshots may be triggered.

In yet another example, a morphology of an ectopic event, that is a morphological shape of a signal portion indicative of a contraction event classified as an ectopic event, may be analyzed, and based on the morphology of the ectopic event a snapshot may be stored. In particular, the storing of a snapshot may be triggered if the morphology of an ectopic event can be classified to match a predefined morphological class, for example because the morphology of the ectopic event sufficiently matches with respect to certain morphological properties a predefined template indicative of a particular morphological class. In another example, the storing of a snapshot may be triggered if an ectopic event has a morphological shape which does not fall into a predefined morphological class, but is of a new, yet unknown morphology not falling into any known class.

For assessing whether an ectopic event falls into a predefined morphological class, in particular certain morphological criteria, such as amplitude parameters, area parameters or timing parameters of a signal portion indicative of the ectopic event may be assessed and may be compared to predefined parameters of a particular morphological class, wherein a match with respect to a morphological class is identified if with respect to the certain parameters deviations are within predefined bounds.

In one embodiment, the processing module may be configured to store the snapshot based on a trend of the occurrence measure over time. In particular, the occurrence measure may be observed over time, and based on a trend of the occurrence measure, that is a change of the occurrence measure over time, it is determined whether to store a snapshot. In particular, the number of ectopic events per hour may be determined as the occurrence measure. A change of this occurrence measure over time, that is over days, weeks or months may be observed, and if it is found that the occurrence measure (i.e. the number of ectopic events per hour) e.g. steadily increases over time or reaches a certain threshold after a steady increase over time, this may trigger the storing of one or multiple snapshots indicative of the time period at and around the threshold crossing.

In one embodiment, the processing module is configured to store the snapshot based on at least one morphological property and/or at least one timing property of an ectopic event. In particular, so-called coupling intervals of the ectopic event to a prior cardiac event and to a subsequent cardiac event may be observed according to timing distances of the ectopic event with respect to the prior cardiac event and with respect to the subsequent cardiac event. If for example it is found for an ectopic event that a timing distance with respect to a prior cardiac event is smaller than a predefined first bound, or if it is found that the timing distance with respect to a subsequent cardiac event is larger than a predefined second bound, this may trigger the storing of a snapshot including a signal portion encompassing the ectopic event.

In one embodiment, the processing module is configured to store a first snapshot based on a number of ectopic events in a first time period, the snapshot encompassing the first time period. Further, the processing module is configured to store a second snapshot to replace the first snapshot based on a number of ectopic events in a second time period subsequent to the first time period if the number of ectopic events in the second time period exceeds the number of ectopic events in the first time period. This may be repeated, such that another snapshot in a another, subsequent time period after the second time period is stored if the number of ectopic events in the other, subsequent time period exceeds the number of ectopic events in the second time period, and so on. Hence, snapshots are continuously overwritten, such that in the end a snapshot of such time period is obtained encompassing the largest number of ectopic events, for example in a larger timeframe such as within an hour, a day or a week. In this way for example in a time frame of 1 hour or 1 day that 1-minute time period may be determined in which the largest number of ectopic events has occurred.

Alternatively or in addition, snapshots may be ranked by priority and may be overwritten according to their priority. For example, a snapshot comprising premature ventricular contractions may have a higher priority than a snapshot comprising (only) premature atrial contractions. A snapshot having a higher priority herein may be used to overwrite a snapshot of a lower priority, which has previously been stored.

Yet alternatively or in addition, a snapshot may be stored based on a timing distance of an ectopic event, for example a timing distance to a prior contraction event. If subsequently in a time period an ectopic event with e.g. a timing distance shorter than the timing distance of the ectopic event of the prior snapshot is detected, a snapshot comprising the ectopic event with the shorter timing distance may overwrite the prior snapshot, such that the snapshot with the shortest timing distance of an ectopic event with respect to a prior contraction event is obtained.

In one embodiment, the implantable medical device is configured to communicate a data representation of the snapshot to an external device. The implantable medical device may be configured to communicate the data representation of the snapshot to the external device immediately upon storing the snapshot. Alternatively or in addition, the implantable medical device may communicate the data representation of the snapshot to the external device within a periodic reporting scheme, such as at a fixed time interval, for example at a predefined time per hour, per day or per week. Alternatively or in addition, the implantable medical device may store the snapshot for a later download or review.

In one embodiment, the processing module is configured to classify a contraction event as an ectopic event based on a first timing distance between the contraction event and an immediately prior contraction event and/or a second timing distance between the contraction event and an immediately subsequent contraction event.

Generally, for an ectopic event, in particular for a premature ventricular contraction event, the morphology of a waveform relating to the premature ventricular contraction event may differ from the morphology of a regular contraction event. This may assessed based on morphological properties. In addition, it may be characteristic for an ectopic event that the contraction event comes at a relatively short timing distance after a prior contraction event and is followed by a lengthy pause before another, regular contraction event occurs. Hence, the timing of the currently assessed contraction event may be evaluated in order to define a condition to classify the currently assessed contraction event as an ectopic event. If for example the first timing distance between the contraction event and the immediately prior contraction event is smaller than a first timing threshold and/or the second timing distance between the contraction event and the immediately subsequent contraction event is larger than a second timing threshold, the currently assessed contraction event may be classified as an ectopic event.

In one embodiment, the processing module is configured to classify the contraction event as an ectopic event based on a morphological measure derived from a signal portion indicative of the contraction event. Alternatively or in addition to assessing timing properties of a contraction event in order to classify the contraction event as an ectopic event, morphological properties may be assessed. This is based on the finding that in particular for premature ventricular contraction events and also for interpolated ventricular contraction events a significant change in morphology may occur, the change in morphology distinguishing the contraction event from a regular contraction event.

In one embodiment, the processing module is configured to classify the ectopic event as a premature ventricular contraction event or a premature atrial contraction event based on the morphological measure. Generally, a premature ventricular contraction event will exhibit a significant change in morphology, in addition to an abnormal timing. In contrast, a premature atrial contraction generally does not significantly differ in its morphological shape from a regular heartbeat, but comes at an abnormal timing. Hence, if it is found that a contraction event comes at an abnormal timing and in addition differs in its morphological shape significantly from a regular heartbeat, the contraction event may be classified as a premature ventricular contraction event. If it in contrast is found that the contraction event comes at an abnormal timing, but does not significantly differ in its morphological shape from a regular heartbeat, the contraction event may be classified as a premature atrial contraction event. Information regarding the classification as a premature ventricular contraction event or as a premature atrial contraction event may be used to trigger the storing of a snapshot, and/or may be stored in addition to the snapshot, such that it for example may be stored that a particular snapshot relates to one or multiple ectopic events classified as premature ventricular contraction events or to one or multiple ectopic events classified as premature atrial contraction events.

In one embodiment, the processing module is configured to compute at least one discrimination metric value for said contraction event, compare said at least one discrimination metric value to at least one of a first reference value computed based on a first number of prior contraction events and a second reference value computed based on a second number of subsequent contraction events, and classify said contraction event as an ectopic event (in particular a premature ventricular contraction event) based on said comparison.

This makes use of the fact that for an ectopic event, in particular a premature ventricular contraction event, it can be assumed that a waveform relating to the ectopic event comprises an abnormal shape, in comparison to other, regular contraction events. Hence, within the implantable medical device, an ectopic event shall be identified by assessing the morphology of a waveform relating to a particular contraction event. If it is found that abnormalities exist in a particular contraction event indicating an ectopic event, the particular contraction event in question shall be classified as an ectopic event.

Hence, one or multiple discrimination metric values are computed. The discrimination metric values relate to the morphology of a waveform of the contraction event which is currently assessed. The one or the multiple discrimination metric values are compared to reference values, and based on the comparison it is identified whether the discrimination metric values indicate an abnormal waveform possibly indicative of an ectopic event (in particular a premature ventricular contraction event).

Herein, the one or the multiple discrimination metric values are compared to one or multiple reference values relating to prior contraction events, which generally can be assumed to be regular contraction events of regular heartbeats, and/or to one or multiple reference values relating to subsequent contraction events, which also generally can be assumed to relate to regular heartbeats. The reference values hence are determined based on prior contraction events and/or subsequent contraction events.

Each reference value should indicate a value for a discrimination metric value in question which is indicative of a normal state and hence a normal contraction event of a regular heartbeat. If, by the comparison of the discrimination metric value computed for the instant contraction event, it is found that the discrimination metric value differs from the reference value e.g. by more than a certain margin, it is identified that an abnormal waveform exists exhibiting an abnormal morphology, such that the contraction event may be classified as an ectopic event (in particular a premature ventricular contraction event).

The first number of prior contraction events (in particular regarding morphology and amplitude), based on which the first reference value is determined, may for example lie in a range between 2 to 50, for example 3 to 20, for example 6 prior contraction events. The prior contraction events may be successive contraction events immediately prior to the instantly assessed contraction event, or may be non-successive.

The second number of subsequent contraction events (in particular regarding morphology and amplitude), based on which the second reference value is determined, may for example lie in a range between 2 to 50, for example 3 to 20, for example 6 subsequent contraction events. The subsequent contraction events may be successive contraction events immediately subsequent to the instantly assessed contraction event, or may be non-successive.

In particular regarding timing only the previous one interval (prior contraction event) and only the following one interval (subsequent contraction event) might be involved. However, timing, amplitude or morphology the criteria should be included: Is the current interval (current contraction event) different than the at least one previous/prior interval (contraction event) and/or the at least one following/subsequent interval (contraction event)?

The first number may be equal to the second number or may differ from the second number.

The classification of an ectopic event generally takes place, in particular if a second reference value relating to subsequent contraction events is taken into account, with a certain time delay to cover a time period within which the subsequent contraction events are recorded.

In one embodiment, the processing module is configured to compute the at least one discrimination metric value, with relation to a signal portion relating to the contraction event, based on at least one of the following: a maximum positive amplitude, a maximum negative amplitude, a maximum rectified amplitude, a peak-to-peak amplitude, a maximum first derivative value, a maximum second derivative value, an area value under a curve of said signal portion until a first zero-crossing, an area value under a curve of said signal portion between a first zero-crossing and a second zero crossing, an area value under a curve of said signal portion between a second zero-crossing and a third zero crossing, a time duration value until a first zero-crossing, a time duration value between a first zero-crossing and a second zero crossing, a time duration value between a second zero-crossing and a third zero crossing and/or a time duration value between an upward crossing and a downward crossing of a ventricular detection threshold.

One or multiple quantities may be computed, accordingly, as one or multiple discrimination metric values. The discrimination metric value may for example be computed according to the maximum positive amplitude, the maximum negative amplitude, a maximum rectified amplitude, or a peak-to-peak amplitude of a waveform relating to a currently assessed contraction event. Alternatively or in addition, a discrimination metric value may be computed according to a maximum value of a first derivative or a second derivative of the waveform relating to the currently assessed contraction event. Alternatively or in addition, the discrimination metric value may be computed according to an area value indicative of an area under a curve relating to the currently assessed contraction event, wherein the area value may relate to the area until the first zero-crossing, to an area between the first zero-crossing and a second zero-crossing, or to an area between the second zero-crossing and a third zero-crossing. Alternatively or in addition, the discrimination metric value may be computed to be indicative of a time duration, for example between the initial detection of the contraction event and a first zero-crossing, between the first zero-crossing and a second zero-crossing, or between the second zero-crossing and a third zero-crossing, or between an upward crossing of a ventricular detection threshold, based on which the ventricular detection event is initially identified, and a downward crossing of the ventricular detection threshold.

Generally, one or multiple discrimination metric values may be computed, wherein any combination of discrimination metric values may be used to identify an ectopic event. Each discrimination metric value herein is compared to at least one of an associated first reference value and an associated second reference value, the particular reference value being indicative of a normal value for the particular discrimination metric value in question.

Each reference value may be computed by applying a statistical analysis of prior contraction events and/or subsequent contraction events. In particular, the first reference value may be computed based on a first statistical measure relating to the first number of prior contraction events. In turn, the second reference value may be computed based on a second statistical measure relating to the second number of subsequent contraction events. The first reference value hence is determined by statistical analysis of prior contraction events. In contrast, the second reference value is determined by statistical analysis of subsequent contraction events.

The particular reference value in particular may be computed according to any standard statistical quantity obtained by statistical analysis. For example, the first reference value and/or the second reference value may be computed according to a mean value, a standard deviation value, a coefficient of variation, a Shannon entropy value, an exponential moving average value e.g. according to a function with varying beats/varying weights associated with the surrounding beats, a median value, a percentile value, e.g. a 5% to 95 % percentile value, a skew value, a kurtosis value, and/or a root mean square value e.g. of successive differences. If for example the discrimination metric value in question is computed according to the maximum amplitude of the waveform relating to the current contraction event, the reference value is determined by e.g. an averaging or by another statistical measure of the maximum amplitude values of prior contraction events and/or subsequent contraction events. If the discrimination metric value in question is an area value relating to the waveform of the current contraction event or a time duration value, the reference value is determined by a statistical measure relating to the associated area value or time duration value of prior contraction events and/or subsequent contraction events.

In one embodiment, the processing module is configured to classify the currently assessed contraction event as an ectopic event (in particular a premature ventricular contraction event) if the at least one discrimination metric value deviates from the first reference value by more than a first margin and/or deviates from the second reference value by more than a second margin. For classifying a currently assessed contraction event as an ectopic event, the processing module compares one or multiple computed discrimination metric values to one or multiple reference values relating to prior contraction events and/or subsequent contraction events. If it is found that the one or the multiple discrimination metric values differ from associated reference values, this is interpreted to indicate that the waveform of the currently assessed contraction event is abnormal in that it differs from a waveform of a regular contraction event, and accordingly the contraction event may be classified as an ectopic event, in particular a premature ventricular contraction event (wherein potentially further conditions may be taken into account).

For example, the processing module is configured to determine the first margin based on a percentage value of the first reference value and/or to determine the second margin based on a percentage value of the second reference value. The first reference value and the second reference value are dynamically determined based on a number of prior contraction events and/or based on a number of subsequent contraction events. Based on the current value for the first reference value and/or the second reference value the first margin and/or the second margin are set. The percentage value herein may be fixed, e.g. in a range between 1% to 50% or the like. In another embodiment the percentage value may be dynamically adapted, for example based on the current value of the first reference value and/or the second reference value.

In one embodiment, the processing module is configured to compute multiple discrimination metric values for the contraction event and to compare the multiple discrimination metric values to multiple first reference values computed based on the first number of prior contraction events and/or multiple second reference values computed based on the second number of subsequent contraction events. Hence, multiple discrimination metric values are determined for the currently assessed contraction event and are compared to associated first and/or the second reference values. Based on the comparison of the multiple discrimination metric values to the associated reference values, the currently assessed contraction event is classified as an ectopic event (or not).

In one embodiment, the processing module is configured to classify the contraction event as an ectopic event (in particular a premature ventricular contraction event) if, based on the comparison, at least for a subset of the discrimination metric values a set of predefined conditions is fulfilled. Within the comparison, the computed discrimination metric values are compared to the associated reference values. If at least for some discrimination metric values the comparison yields that the discrimination metric values for example differ from the associated reference values in each case by more than a certain margin, the currently assessed contraction event may be classified as a premature ventricular contraction event. For example, it may be generally required that for two out of three discrimination metric values the comparison yields that the discrimination metric values differ from the associated reference values by more than a certain margin, such that for two out of three discrimination metric values an associated condition is fulfilled.

The implantable medical device may, in one embodiment, comprise multiple electrode poles, which for example are aligned along a longitudinal axis and hence are arranged at different axial positions on the implantable medical device. By means of the different electrode poles an electrical coupling to surrounding tissue is established when the implantable medical device is implanted in a patient, such that electrocardiogram signals may be sensed using the different electrode poles.

In another aspect, a method for operating an implantable medical device for sensing electrocardiogram signals comprises: sensing electrocardiogram signals using an arrangement of electrode poles of the implantable medical device; and processing electrocardiogram signals obtained by the arrangement of electrode poles using a processing module of the implantable medical device. The processing module identifies a contraction event based on said electrocardiogram signals, classifies said contraction event as an ectopic event based on a relation to other contraction events prior and/or subsequent to said contraction event, and stores a snapshot representing data (e.g. a signal portion) of said electrocardiogram signals in a time period encompassing a contraction event classified as an ectopic event.

The advantages and advantageous embodiments described above for the system equally apply also to the method, such that it shall be referred to the above in this respect.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The various features and advantages of the present invention may be more readily under-stood with reference to the following detailed description and the embodiments shown in the drawings. Herein, FIG. 1 shows a schematic drawing of an implantable medical device implanted in a patient;

FIG. 2 shows a schematic drawing of an embodiment of an implantable medical device comprising an arrangement of electrode poles;

FIG. 3 shows a schematic drawing of another embodiment of an implantable medical device;

FIG. 4 shows electrocardiogram signals relating to ectopic events;

FIG. 5 shows a signal waveform relating to a premature ventricular contraction event;

FIG. 6 shows a series of contraction events; and

FIG. 7 shows a premature ventricular contraction event within a series of contraction events.

### DETAILED DESCRIPTION

Subsequently, embodiments of the invention shall be described in detail with reference to the drawings. In the drawings, like reference numerals designate like structural elements.

It is to be noted that the embodiments are not limiting for the invention, but merely represent illustrative examples.

Referring to FIG. 1, in one embodiment a system comprises at least one implantable medical device 1 implanted (for example subcutaneously) into a patient for serving a therapeutic and/or diagnostic function. The implantable medical device 1 may for example be implanted subcutaneously into a patient P for monitoring cardiac activity of the patient's heart H. Further implantable medical devices (not shown), for example an implantable pulse generator (IPG) and/or an implantable cardiac defibrillator (ICD) may be implanted into the patient P to provide further (ECG) data. Such further implantable medical devices may be implanted subcutaneously) with a wire (lead with at least one electrode pole) going into the heart or two wires (leads each with at least one electrode pole), one going into the atrium and one going into the ventricle of the heart. The implantable medical device 1, for this, comprises an arrangement of electrode poles which are used to couple to surrounding tissue and to sense electrocardiogram signals originating from the heart H.

The system furthermore comprises an external device 2 external to the patient P and being in communication connection with the implantable medical device 1.

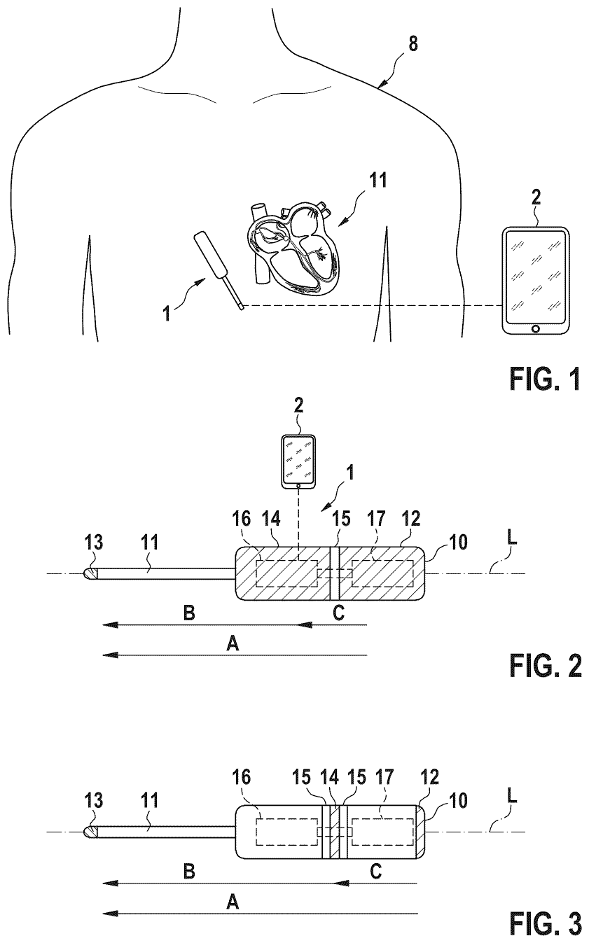

Referring now to FIG. 2, in one embodiment the implantable medical device 1 comprises a housing 10 formed e.g. by different housing segments, the housing 10 enclosing and encapsulating a processing module 16 formed by electronic circuitry and a battery module 17. In particular, a first housing segment may receive and enclose the processing module 16, whereas a second housing segment receives and encloses the battery module 17. Another housing segment longitudinally extends from the first and second housing segments and forms a header portion 11 having reduced cross-sectional dimensions with respect to the other housing segments.

In the embodiment of FIG. 2, a first electrode pole 12 is formed by the housing segment enclosing the battery module 17, a second electrode pole 13 is arranged at a far end of the housing segment forming the header portion 11, and (optional) a third electrode pole 14 is formed by the housing segment enclosing the processing module 16. However, most important are the two electrode poles 12, 13 that are the farthest apart. The implantable medical device 1 with its housing 10 generally extends along a longitudinal axis L, the electrode poles 12, 13, 14 being aligned along the longitudinal axis L and being axially displaced with respect to one another along the longitudinal axis L. The electrode poles 12, 13, 14 herein are electrically separated from one another, an electrically insulating segment 15 being arranged in between the electrode poles 12, 14 formed on the main housing portion and the header portion 11 formed by the housing segment separating the electrode pole 13 from the other two electrode poles 12, 14.

In the embodiment of FIG. 2, the electrode poles 12, 13, 14 may be formed by portions of the housing 10 itself, the housing 10 being made for example from an electrically conductive material, in particular a metal material. By exposing portions of the housing 10 towards the outside, the electrode poles 12, 13, 14 are formed and may electrically contact with surrounding tissue in order to establish a coupling between the electrode poles 12, 13, 14 to the surrounding tissue.

Referring now to FIG. 3, in another embodiment the first electrode pole 12 is formed at an end of a housing segment of the housing 10 encapsulating the battery module 17, whereas the (optional) electrode pole 14 is formed by an electrode element which is electrically insulated from other portions of the housing 10 by the electrically insulating segments 15. Here again, most important are the two electrode poles 12, 13 that are the farthest apart. For example, a multilayered pole element may be employed for forming the electrode pole 14, as it is described for example in EP 3 278 836 B1. The electrode pole 13 again is formed at a far end of the housing segment forming the header portion 11.

In any of the embodiments of FIGS. 2 and 3, using the arrangement of electrode poles 12, 13 and optional 14, electrocardiogram signals may be received and processed by the processing module 16. Based on the processing, a communication with an external device 2 may be established, for example to transmit alert messages to the external device 2 for example within the context of a home monitoring system for monitoring a physiological state of the patient P.

The different electrode poles 12, 13 and optional 14 herein define at least one signal reception vector A and optionally reception vectors B, C by means of which electrocardiogram signals may be received using pairs of associated electrode poles 12, 13, 14. In particular, a first signal reception vector A is formed between the first electrode pole 12 and the second electrode pole 13, a second signal reception vector B is formed between the third electrode pole 14 and the first second electrode pole 13, and a third signal reception vector C is formed between the first electrode pole 12 and the third electrode pole 14. As the first electrode pole 12 and the second electrode pole 13 are arranged at opposite ends of the housing 10, the associated signal reception vector A is longer than the other two signal reception vectors B, C.

The different electrode poles 12, 13, 14 form different pairs of electrode poles 12, 13, 14 spanning different signal reception vectors A, B, C. By means of the different signal reception vectors A, B, C different electrocardiogram signals may be received and may be processed in a multi-channel processing.

It shall be noted herein that the implantable medical device 1 may be a monitoring device (as schematically shown in FIGS. 2 and 3), a pacemaker device, a defibrillator device or any other implantable medical device configured for implantation into a patient P. The instant text in particular is not limited to a monitoring device configured for implantation outside of a patient's heart H.

The implantable medical device 1 as described herein shall generally be configured to identify ectopic events.

Generally, it is characteristic for ectopic events, such as a premature ventricular contraction event, that the ectopic event comes at a premature, short timing distance after a prior contraction event and is followed by a comparatively lengthy pause before another, subsequent contraction event occurs. In addition, an ectopic event such as a premature ventricular contraction event generally exhibits a waveform which in its morphology substantially differs from the waveform of a regular contraction event.

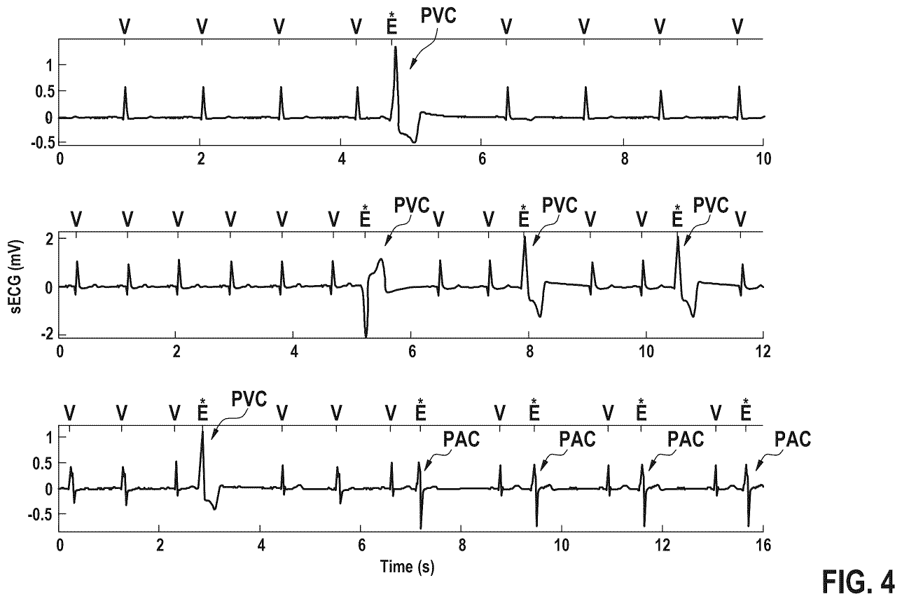

Referring now to FIG. 4, ectopic events E* may be distinguished from regular contraction events V of a regular sine rhythm according to a timing and in addition according to an analysis of a morphology of a particular contraction event. FIG. 4 herein shows different time series of electrocardiogram data signals, each of which containing contraction events V according to regular heartbeats and in addition one or multiple ectopic events E *.

In the example of the electrocardiogram signal at the top of FIG. 4, one ectopic event E* is identified within a series of contraction events V of a regular sine rhythm, the ectopic event E* being classified according to its abnormal timing and its abnormal morphological shape as a premature ventricular contraction event PVC.

In the example of the middle row of FIG. 4, in turn, multiple ectopic events E* are identified within a series of contraction events V, each ectopic event E* being classified as a premature ventricular contraction event PVC. The ectopic events E* herein differ in their morphological shape, a first ectopic event within the time series having a substantially different morphological shape than subsequent ectopic events E *.

In yet another example according to the bottom row of FIG. 4, different ectopic events E* are present, wherein a first ectopic event E* is classified as a premature ventricular contraction event PVC, whereas further ectopic events E* are classified as premature atrial contraction events PAC. As visible from the bottom row of FIG. 4, the premature ventricular contraction event PVC and the premature atrial contraction events PAC both exhibit an abnormal timing with respect to contraction events V of a regular sine rhythm.

However, whereas the morphological shape of the premature ventricular contraction event PVC substantially differs from the morphology of a regular contraction event V, the premature atrial contraction events PAC are at least to some extent morphologically similar to regular contraction events V, for example with respect to the amplitude of an R wave and with respect to zero-crossings of a related waveform.

The processing module 16 is configured to identify one or multiple ectopic events E* in electrocardiogram signals. Electrocardiogram signals herein may be obtained by an arrangement of one or multiple pairs of electrode poles, such that a one-channel or multi-channel processing of electrocardiogram signals may be carried out.

The processing module 16 of the implantable medical device 1 herein is configured to store a snapshot representing data (e.g. a signal portion, sample time series data, and/or trends of intervals or amplitudes) of electrocardiogram signals in a time period encompassing a contraction event V classified as an ectopic event E *. The length of the time period may be predefined, e.g. programmed by a user, and may for example have a length in between 10 seconds to 24 hours, for example in between 30 seconds to 10 minutes, for example 1 minute.

Hence, in the example of the top row of FIG. 4, if an ectopic event E* is identified, a snapshot may be stored, the snapshot containing a time series of electrocardiogram signal data encompassing the ectopic event E* and prior and subsequent regular contraction events V.

Thus, the detection of one or multiple ectopic events E* triggers the storing of a snapshot. A data representation of the snapshot may be communicated to an external device 2, such that the snapshot may be assessed by a physician in order to evaluate the occurrence of ectopic events E* to conclude for potential countermeasures and treatment options. A communication of a snapshot herein may take place immediately upon storing a snapshot. In addition or alternatively, a snapshot may be communicated by the implantable medical device 1 within a periodic reporting, for example at a fixed time of day or the like. Yet alternatively or in addition, a snapshot may be stored within the implantable medical device 1 for future download and review by a user.

Each occurrence of an ectopic event E* may generally trigger the storing of a snapshot. Alternatively, a storing of a snapshot may be triggered only if a certain trigger condition is met.

For example, a storing of a snapshot may be triggered if a number of ectopic events E* in a certain time interval, for example in a minute or in an hour, exceeds a predefined threshold. Hence, a snapshot is taken if a number of ectopic events E* occurs in a time interval which is larger than a predefined threshold.

In another example, timing properties or morphological properties may be assessed. For example, the timing distance of an ectopic event E* with respect to a prior contraction event V or a subsequent contraction event V may be assessed. If for example the timing distance with respect to the prior contraction event V is smaller than a predefined amount, or if it is found that the timing distance with respect to a subsequent contraction event V is larger than a predefined bound, this may trigger the storing of a snapshot.

In yet another example, the morphology of ectopic events E* may be assessed. For example, templates of morphological classes may be predefined. If it is found that an ectopic event E* falls into a predefined morphological class in that the shape of the ectopic event E* within the electrocardiogram signal with respect to certain morphological properties substantially matches the predefined template, this may trigger the storing of a snapshot.

Also, if it is found that an ectopic event E* does not match a predefined morphological class and hence is associated with a yet unknown type of ectopic event E*, this may trigger the storing of a snapshot.

Also, if a change in morphology, as visible in the middle row of FIG. 4, occurs, this may trigger the storing of a snapshot encompassing the various morphological shapes of the ectopic events E *.

In the identification of the ectopic events E*, it may be differentiated between premature ventricular contraction events PVC and premature atrial contraction events PAC, as visible in the bottom row of FIG. 4. Based on a classification of ectopic events E* as premature ventricular contraction events PVC or premature atrial contraction events PAC, a storing of a snapshot may be triggered. As premature ventricular contractions PVC generally are assumed to have a larger impact on a cardiac state of a patient, snapshots containing premature ventricular contraction events PVC may be given a higher priority in comparison to snapshots containing premature atrial contraction events PAC, wherein a snapshot of a higher priority may overwrite a snapshot of a lower priority.

Ectopic events E* may be identified continuously in time periods. A first snapshot herein may be taken in a first time period containing one or multiple ectopic events E*. If in a later time period it is found that the number of ectopic events E* exceeds the number of ectopic events E* in the first time period, a snapshot may be stored and may replace the prior snapshot. This may be continued, such that in the end a snapshot representative of the time period with the largest number of ectopic events E* is obtained within a predefined overall timeframe, for example within an hour or a day, which then may be reported to an external device 2.

Ectopic events E* may generally occur as premature ventricular contraction events PVC, premature atrial contraction events PAC or so-called interpolated premature ventricular contraction events. A classification of a contraction event as an ectopic event generally may take place based on an assessment of timing distances with respect to other contraction events, and based on an analysis of morphological properties. Ectopic events in particular may be identified based on an abnormal timing in combination with morphological characteristics.

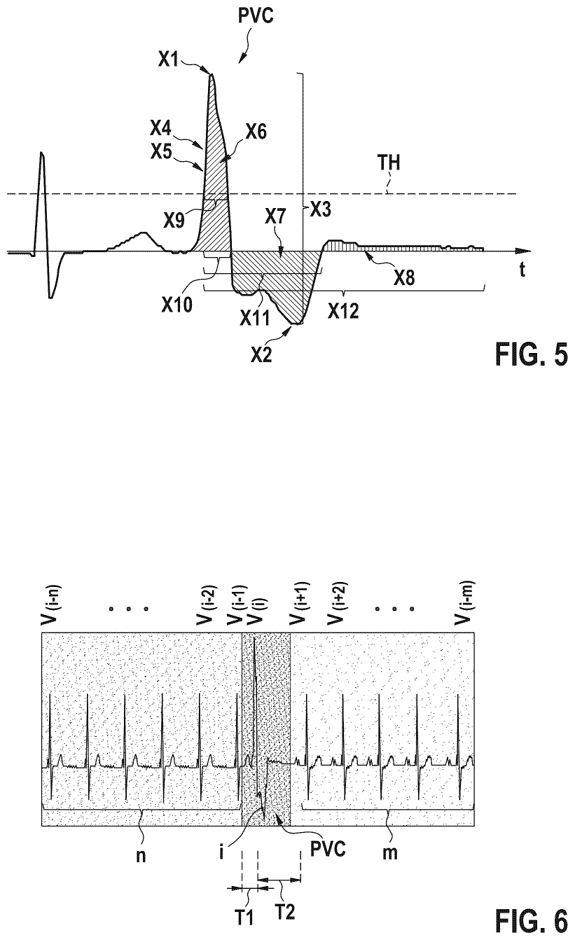

Referring now to FIG. 5, an ectopic event such as a premature ventricular contraction event PVC generally comprises a morphology different than a regular contraction event of a regular sine rhythm of the patient's heart H. The morphology herein may be characterized by certain discrimination metrics, such as a maximum positive amplitude X1, a maximum negative amplitude X2, a maximum peak-to-peak amplitude X3, a maximum slope value X4, an maximum value of the second derivative X5, an area X6 under a positive R peak prior to a first zero-crossing X6, an area X7 under the curve between a first zero-crossing and a second zero-crossing, an area X8 under the curve between the second zero-crossing and a third zero-crossing, a time duration X9 between an upward crossing and a downward crossing of a ventricular detection threshold TH, a time duration X10 between the upward crossing of the ventricular detection threshold TH and the first zero-crossing, a time duration X11 between the upward crossing of the ventricular detection threshold TH and the second zero-crossing, and/or a time duration X12 between the upward crossing of the ventricular detection threshold TH and a third zero-crossing.

For a particular ventricular contraction waveform in question, values for all or some of the discrimination metrics may be computed and may be assessed in order to identify whether an abnormal morphology potentially indicative of an ectopic event such as a premature ventricular contraction event is present.

To classify at least one contraction event as an ectopic event such as a premature ventricular contraction event, in one embodiment at least one discrimination metric value X1 . . . X12 is computed by the processing module 16 of the implantable medical device 1. The at least one discrimination metric value is then compared to at least one reference value, and based on the comparison the contraction event in question is classified as an ectopic event such as a premature ventricular contraction waveform (or not).

Referring now to FIG. 6, reference values may in particular be determined according to a number n of prior contraction events V(i−n) . . . V(i−1) and/or a number m of subsequent contraction events V(i+1) . . . V(i+m). In particular, for a specific discrimination metric value, such as the maximum positive amplitude X1 or the area X6 under the positive R peak prior to the first zero-crossing, an associated first reference value may be computed based on the associated discrimination metric for the number n of prior contraction events V(i−n) . . . V(i−1). Alternatively or in addition, a second reference value may be computed based on the associated discrimination metric for the number m of subsequent contraction events V(i+1) . . . V(i+m).

The particular reference value may in particular be computed according to a statistical measure by applying a statistical analysis. For example, the particular reference value may correspond to a mean value, a standard deviation value, a coefficient of variation, a Shannon entropy value, an exponential moving average value, a median value, a percentile value, a skew value, a kurtosis value, or a root mean square value relating to the particular discrimination metric X1 . . . X12.

For example, if the maximum positive amplitude X1 is assessed as the discrimination metric, the first reference value may be determined by averaging the maximum positive amplitude values of the n prior contraction events V(i−n) . . . V(i−1), and the second reference value may be determined by averaging the maximum positive amplitude values of the m subsequent contraction events V(i+1) . . . V(i+m).

Making use of the reference values, then, the particular contraction event in question may be classified as an ectopic event such as a premature ventricular contraction event if for example a particular discrimination metric value as computed for the contraction event differs by more than a certain margin from the respective reference value. For example, if both a first reference value relating to prior contraction events V(i−n) . . . V(i−1) and a second reference value relating to subsequent contraction events V(i+1) . . . V(i+m) is taken into account, a contraction event may be classified as an ectopic event such as a premature ventricular contraction event if the discrimination metric value differs by more than a first margin from the first reference value and by more than a second margin from the second reference value.

If multiple discrimination metric values relating to different discrimination metrics are computed, multiple different first reference values and/or or multiple different second reference values may be taken into account, the different reference values relating to the different discrimination metrics. Herein, the contraction event may be classified as an ectopic event such as a premature ventricular contraction event if at least for a subset of the discrimination metric values an associated set of conditions is fulfilled. For example, it may be found for an ectopic event such as a premature ventricular contraction event if for two out of three of the discrimination metric values it is found that the particular discrimination metric value differs from an associated reference value by more than a certain margin.

The margin, in each case, may be for example computed based on a percentage of the particular reference value, wherein the percentage may be fixed or may be dynamically adapted during operation of the system.

In the example of FIG. 6, for the contraction event V(i) it for example is found that based on one or multiple discrimination metric values relating to one or multiple different discrimination metrics a substantial deviation of the morphology in comparison to prior ventricular events V(i−n) . . . V(i−1) and/or to subsequent ventricular events V(i+1) . . . V(i+m) is present, such that the contraction event V(i) is classified as an ectopic event such as a premature ventricular contraction event PVC.

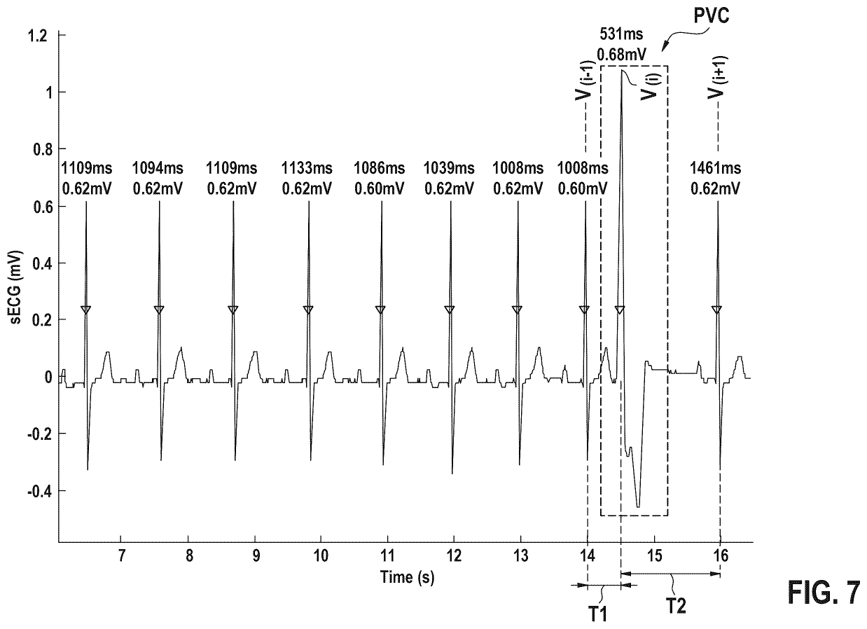

Generally, an ectopic event differs in the timing from a regular contraction event. Hence, it may be assessed whether a timing distance Tl of the contraction event V(i) is smaller than a first timing threshold, hence indicating that the contraction event V(i) occurs prematurely with respect to a prior contraction event V(i−1), as indicated in FIG. 7. In addition, it may be assessed whether a timing distance T2 of the contraction event V(i) is larger than a second timing threshold, hence indicating that after the contraction event V(i) a substantial pause occurs, longer than the length of a regular heartbeat, as visible in FIG. 7.

In order to provide for a reliable classification of an ectopic event such as, hence, a timing and in addition a morphology may be assessed, wherein the combined assessment yields a classification of a current contraction event V(i) as an ectopic event if both a timing condition and a morphology condition is fulfilled.

For example, in one embodiment, an R-wave peak amplitude discrimination metric is used in combination with timing. If the peak amplitude of a given QRS complex is different from the mean of previous peak amplitudes by a given percentage and is also different from the mean of the following R-wave peak amplitudes by a second given percentage, and the interval preceding the given QRS complex is shorter than a given short interval threshold and the interval following the QRS complex is longer than a given long interval threshold, then the QRS complex is classified as an ectopic event such as a premature ventricular contraction event PVC. In this embodiment, the minimum change in R-wave amplitude may fall in the range of 2.5% to 50% of the preceding or following cycles' R-wave amplitude. The timing threshold for determining the short or premature interval may be determined in either a number of milliseconds or a percentage or the instantaneous or average cycle time. Furthermore, the threshold may be static as a programmable fixed number or could be a dynamic parameter that is adjusted based on the variation in the RR-intervals in the vicinity of the current QRS complex. The same principles may apply to the determination of the long interval threshold. The amplitude threshold may also be a static percentage (like 10%) or may be dynamically determined based on the amount of variation in previous or following signal amplitudes.

In another embodiment, similar principles are applied but a different discrimination metric is used. In this embodiment the critical quantity is the time-to-zero crossings. The individual parameters, or a combination of zero-crossing times X10, X11, X12 determined as the weighted sum of related time periods X10, X11, X12 are used as discrimination metrics in order to determine if the morphology and/or the total duration of the complex has changed. This final combination may be used alone or together with timing criteria as described in the first embodiment. Absolute thresholds or dynamic thresholds based on the properties of the signals from past cycles or next cycles may be used to determine if the instant contraction event is an ectopic event such as a premature ventricular contraction event PVC.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Implantable medical device
10 Housing
11 Header portion
12 First electrode pole
13 Second electrode pole
14 Third electrode pole
15 Electrically insulating segment
16 Processing module
17 Battery module
2 External device
A, B, C Signal reception vector
E* Ectopic event
H Heart
i Current contraction event (beat)
L Longitudinal axis
n Prior contraction events (beats)
m Subsequent contraction events (beats)
P Patient
PAC Premature atrial contraction event
PVC Premature ventricular contraction event
T1 Timing distance
T2 Timing distance
TA Period of activity
TH Ventricular detection threshold
V(i−n) . . . V(i+m) Contraction event
X1 . . . X12 Discrimination metric value

1. An implantable medical device for sensing electrocardiogram signals, comprising:
an arrangement of electrode poles configured to sense electrocardiogram signals; and
a processing module for processing electrocardiogram signals obtained by the arrangement of electrode poles;
wherein the processing module is configured to identify a contraction event based on said electrocardiogram signals,
classify said contraction event as an ectopic event based on a relation to other contraction events prior and/or subsequent to said contraction event, and
store a snapshot representing data of said electrocardiogram signals in a time period encompassing a contraction event classified as an ectopic event.

2. The implantable medical device according to claim 1, wherein said time period has a length in between 10 seconds and 24 hours, in particular in between 30 seconds and 10 minutes, for example 1 minute.

3. The implantable medical device according to claim 1, wherein the processing module is configured to derive at least one occurrence measure indicative of an occurrence of at least one ectopic event and to store said snapshot based on said at least one occurrence measure.

4. The implantable medical device according to claim 3, wherein the processing module is configured to derive said at least one occurrence measure to be indicative of the occurrence of at least one ectopic event in a predefined time interval, the occurrence of at least one further ectopic event after a prior ectopic event in a predefined time interval, the occurrence of a number of ectopic events in a predefined time interval equal to or larger than a predefined threshold, and/or the occurrence of an ectopic event of a predefined morphological class or of a shape different than a predefined morphological class.

5. The implantable medical device according to claim 3, wherein the processing module is configured to store said snapshot based on a trend of said occurrence measure over time.

6. The implantable medical device according to claim 1, wherein the processing module is configured to store said snapshot based on at least one morphological property and/or at least one timing property of an ectopic event.

7. The implantable medical device according to claim 1, wherein the processing module is configured to store a first snapshot based on a number of ectopic events in a first time period, and store a second snapshot to replace the first snapshot based on a number of ectopic events in a second time period subsequent to said first time period if the number of ectopic events in said second time period exceeds the number of ectopic events in said first time period.

8. The implantable medical device according to claim 1, wherein the implantable medical device is configured to communicate a data representation of said snapshot to an external device.

9. The implantable medical device according to claim 1, wherein the processing module is configured to classify said contraction event as an ectopic event based on a first timing distance between the contraction event and an immediately prior contraction event and/or a second timing distance between the contraction event and an immediately subsequent contraction event

10. The implantable medical device according to claim 1, wherein the processing module is configured to classify said contraction event as an ectopic event based on a morphological measure derived from a signal portion indicative of said contraction event.

11. The implantable medical device according to claim 10, wherein the processing module is configured to classify said ectopic event as a premature ventricular contraction event or a premature atrial contraction event based on said morphological measure.

12. The implantable medical device according to claim 1, wherein the processing module is configured to compute at least one discrimination metric value for said contraction event, compare said at least one discrimination metric value to at least one of a first reference value computed based on a first number of prior contraction events and a second reference value computed based on a second number of subsequent contraction events, and classify said contraction event as an ectopic event based on said comparison.

13. The implantable medical device according to claim 12, wherein the processing module is configured to compute said first reference value based on a first statistical measure relating to said first number of prior contraction events and/or compute said second reference value based on a second statistical measure relating to said second number of subsequent contraction events.

14. The implantable medical device according to claim 12, wherein the processing module is configured to classify said contraction event as an ectopic event if said at least one discrimination metric value deviates from said first reference value by more than a first margin and/or deviates from said second reference value by more than a second margin.

15. A method for operating an implantable medical device for sensing electrocardiogram signals, the method comprising:
sensing electrocardiogram signals using an arrangement of electrode poles of the implantable medical device; and
processing electrocardiogram signals obtained by the arrangement of electrode poles; using a processing module of the implantable medical device;
wherein the processing module identifies a contraction event based on said electrocardiogram signals,
classifies said contraction event as an ectopic event based on a relation to other contraction events prior and/or subsequent to said contraction event, and
stores a snapshot representing data of said electrocardiogram signals in a time period encompassing a contraction event classified as an ectopic event.
